(19) United States
(12) Patent Application Publication
(10) Pub. No.: US 2021/0252072 A1
(43) Pub. Date: Aug. 19, 2021

(54) ENGINEERED HEMATOPOIETIC STEM CELLS FOR THE TREATMENT OF ACUTE MYELOID LEUKEMIA

(71) Applicants: LOMA LINDA UNIVERSITY, Loma Linda, CA (US); LOMA LINDA UNIVERSITY MEDICAL CENTER, Loma Linda, CA (US); FACULTY PHYSICIANS AND SURGEONS OF LOMA LINDA UNIVERSITY SCHOOL OF MEDICINE, Loma Linda, CA (US)

(72) Inventors: Huynh CAO, Loma Linda, CA (US); Yi XU, Loma Linda, CA (US); Kimberly PAYNE, Loma Linda, CA (US); David BAYLINK, Loma Linda, CA (US)

(21) Appl. No.: 16/973,108

(22) PCT Filed: Jun. 13, 2019
(86) PCT No.: PCT/US19/36912

§ 371 Date: Dec. 8, 2020

Related U.S. Application Data
(60) Provisional application No. 62/685,228, filed on Jun. 14, 2018.

(51) Int. Cl. A61K 35/28; C12N 5/0789

(52) U.S. Cl. CPC A61K 35/28; A61K 2035/124; C12N 5/0647

(57) ABSTRACT

In one aspect, engineered hematopoietic stem cells comprising a heterologous expression cassette are provided. In some embodiments, the expression cassette comprises a promoter operably linked to a polynucleotide that encodes a 1α-hydroxylase protein, wherein the 1α-hydroxylase protein is human cytochrome P450 family 27 subfamily B member 1 (CYP27B1).

### BACKGROUND OF THE INVENTION

Acute myeloid leukemia (AML) is a blood cancer that primarily affects older adults. Conventional treatment for AML has been mainly focused on delivering cytotoxic effects to leukemic blasts via chemotherapy. However, many older patients cannot tolerate this intensive therapeutic regimen. For patients who are precluded from receiving standard induction chemotherapy, for example due to advanced age, the outcome is poor, with a median survival of 5-10 months. Dohner et al., N Engl J Med, 2015, 373:1136-1152. Accordingly, there remains a need for methods and compositions for the treatment of AML.

### BRIEF SUMMARY OF THE INVENTION

In one aspect, engineered hematopoietic stem cells are provided. In some embodiments, the engineered hematopoietic stem cell comprises a heterologous expression cassette, the expression cassette comprising a promoter operably linked to a polynucleotide that encodes a 1α-hydroxylase protein, wherein the 1α-hydroxylase protein is human cytochrome P450 family 27 subfamily B member 1 (CYP27B1).

In some embodiments, the promoter is a constitutively active promoter. In some embodiments, the promoter is a SFFV promoter, a PGK promoter, an EF1α promoter, or a CMV promoter. In some embodiments, the promoter is an inducible promoter. In some embodiments, the promoter is a tissue-specific promoter.

In some embodiments, the hematopoietic stem cell is human. In some embodiments, the hematopoietic stem cell is a cord blood-derived cell. In some embodiments, the hematopoietic stem cell is a bone marrow-derived cell. In some embodiments, the hematopoietic stem cell is obtained from a subject who has been treated with 5-azacytidine.

In some embodiments, the engineered hematopoietic stem cell overexpresses CYP27B1. In some embodiments, the engineered hematopoietic stem cell produces at least 10-fold, at least 25-fold, at least 50-fold, at least 75-fold, or at least 100-fold the concentration of active 1,25-VD3 as compared to a hematopoietic stem cell lacking the heterologous expression cassette.

In some embodiments, the engineered hematopoietic stem cell is stimulated with one or more cytokines.

In some embodiments, the engineered hematopoietic stem cell comprises a vector that comprises the heterologous expression cassette. In some embodiments, the vector is a lentiviral vector.

In another aspect, pharmaceutical compositions comprising a population of engineered hematopoietic stem cells as disclosed herein are provided. In some embodiments, the pharmaceutical composition comprises the population of engineered hematopoietic stem cells and further comprises a pharmaceutically acceptable excipient.

In yet another aspect, therapeutic methods comprising the engineered hematopoietic stem cells and pharmaceutical compositions comprising a population of engineered hematopoietic stem cells are provided. In some embodiments, the method is a method of treating a human subject having a leukemia. In some embodiments, the method comprises administering to a subject a population of engineered hematopoietic stem cells or a pharmaceutical composition comprising a population of engineered hematopoietic stem cells, wherein the engineered hematopoietic stem cells comprise a heterologous expression cassette that comprises a promoter operably linked to a polynucleotide that encodes a human CYP27B1 protein.

In some embodiments, the promoter is a constitutively active promoter. In some embodiments, the promoter is a SFFV promoter, a PGK promoter, an EF1a promoter, or a CMV promoter. In some embodiments, the promoter is an inducible promoter. In some embodiments, the promoter is a tissue-specific promoter.

In some embodiments, the leukemia is acute myeloid leukemia (AML). In some embodiments, the AML is AML subtype M0, M1, M2, M4, M5, M6, or M7. In some embodiments, the AML comprises a mutation in FLT3. In some embodiments, the AML comprises an internal tandem duplication in FLT3 (ITD-FLT3) and/or a point mutation in FLT3.

In some embodiments, the subject is an adult. In some embodiments, the subject is a juvenile. In some embodiments, the subject the subject has previously been treated with 5-azacytidine within one day of administering the engineered hematopoietic stem cells.

In some embodiments, the engineered hematopoietic stem cells are autologous to the subject. In some embodiments, the autologous cells are obtained from the subject after treatment with 5-azacytidine. In some embodiments, the engineered hematopoietic stem cells are allogeneic to the subject.

In some embodiments, the engineered hematopoietic stem cells are stimulated with one or more cytokines.

In some embodiments, the engineered hematopoietic stem cells are administered systemically. In some embodiments, the engineered hematopoietic stem cells are administered by infusion or by injection.

### BRIEF DESCRIPTION OF THE DRAWINGS

FIG. 1. Differentiation of AML cells in response to cell-mediated delivery of CYP27B1 enzyme. Mesenchymal progenitor cells carrying the CYP27B1 gene were cultured without VD3 (left panel), with different concentrations of 25-VD3 (inactive substrates for the CYP27B1 gene, middle panels) or with active VD3 as a positive control (right panel). MOLM14 cells were collected at 48 hours and assessed by flow cytometry for expression of the differentiation marker CD14. MOLM14 AML cells showed a dose-dependent increase in CD14 expression.

FIGS. 2A-2C. Synergistic effect of VD3 and AZA combination treatment. MOLM14 and primary human AML cells were treated with active VD3 and AZA alone, and in combination for 48 hours. (A) At the end of treatment, MOLM14 cells were assayed by flow cytometry for expression of the CD14 differentiation marker and staining with viability dye. Combination treatment showed significantly fewer viable blasts (Quadrant 4, and graphed in the inset). (B) Cell cycle analysis of MOLM14 following treatment showed that active VD3 inhibited DNA synthesis while AZA increased apoptosis, and combination treatment increased both. (C) Ex vivo data for 5 AML patient samples with different AML subtype, cytogenetics and molecular mutations. Combination treatment resulted in the largest reduction of blasts in all 5 patients.

FIGS. 3A-3C. Establishment of CYP27B1-luciferase-GFP transduced MOLM14 cells. To help trace live MOLM14 cells in vivo, a new lentiviral construct of CYP27B1-luciferase-GFP was constructed. After highly efficient viral transduction into MOLM14 cells, the CYP27B1-luciferase-GFP transduced MOLM14 (CLGM14) cell line was generated. Next, CLGM14 cells were functionally tested in vitro. (A) D-luciferin was added to CLGM14 cells to test luciferase activity. CLGM14 cells converted the substrate and generated the bioluminescence. (B) Further, CLGM14 cells were observed displayed green fluorescence (GFP) under microscopy. (C) 25-VD3 was added to CLGM14 cells to induce cell differentiation. Flow cytometry shows more CD14+(a monocyte marker) MOLM14 cells with 25-VD3, as compared to fewer CD14+ cells in the control (no addition of 25-VD3).

FIG. 4. The MTT assay reveals the synergistic effect of combination therapy. HL-60 cell (upper panels) and MOLM-14 cells (lower panels) were cultured in 96-well plates and treated with various combinations of VIDAZA® (azacytidine) and active VD3 for 48 hours. The MTT assay was used to measure the antiproliferative effects of treatment and the Combination Index (CI) was calculated to evaluate synergy (synergy=CI<1). Synergistic treatment conditions are shown in bolded red text.

FIG. 5. Generation of patient-derived HSCs overexpressing CYP27B1-LUC-GFP in vitro. Top Panels: Human CD34+ cells were isolated from AML patient peripheral blood using CD24 MicroBead Kit and MACS Separator (Miltenyi Biotec), according to the manufacturer's protocol. Lower Panels: CYP27B1-LUC-GFP viral transduction was performed, resulting in 62.3% CYP-GFP+CD34+ HSCs by FACS analysis.

FIG. 6. MV4-11 AML cell line was assayed by flow cytometry for expression of the CD14 differentiation marker and staining with viability dye. Combination treatment significantly reduced the percentage of viable blasts (Viable/CD14− cells in the gating strategy) from 95.5% with no treatment (p<0.05), and 74.8% with 5 uM AZA alone (p<0.05) to 34.1%. 80 nM 1,25-D3 therapy alone reduced more blasts compared to 5 uM AZA alone by 38.5% vs 74.8% (p<0.05).

FIG. 7. A FLT3-ITD patient primary cell sample was assayed by flow cytometry for expression of the CD14 differentiation marker and staining with viability dye. Combination treatment with 5 uM VIDAZA®+80 nM VD3 showed the most significant reduction of blast cells compared to the controls, from 68.2% with no treatment (p<0.05), 61.8% with 80 nM 1,25-D3 alone (p<0.05), and 42.6% with 5 uM AZA alone to 36.6% (p<0.05).

### DETAILED DESCRIPTION OF THE INVENTION

### I. Introduction

Disclosed herein are compositions and methods of differentiation therapy for the treatment of acute myeloid leukemia (AML). It has previously been shown that AML cells undergo differentiation after exposure to active vitamin D (VD3) in vitro. However, the clinical success of this approach is limited by systemic hypercalcemia from high dose VD3. As disclosed herein, it has been found that hematopoietic stem cells can be engineered to overexpress 1α-hydroxylase in order to produce high local levels of active VD3 in situ to promote differentiation of leukemic blasts. Without being bound to a particular theory, it is believed that adoptive therapy with these engineered hematopoietic stem cells achieves delivery of high concentrations of VD3 to a local target without off-target hypercalcemia.

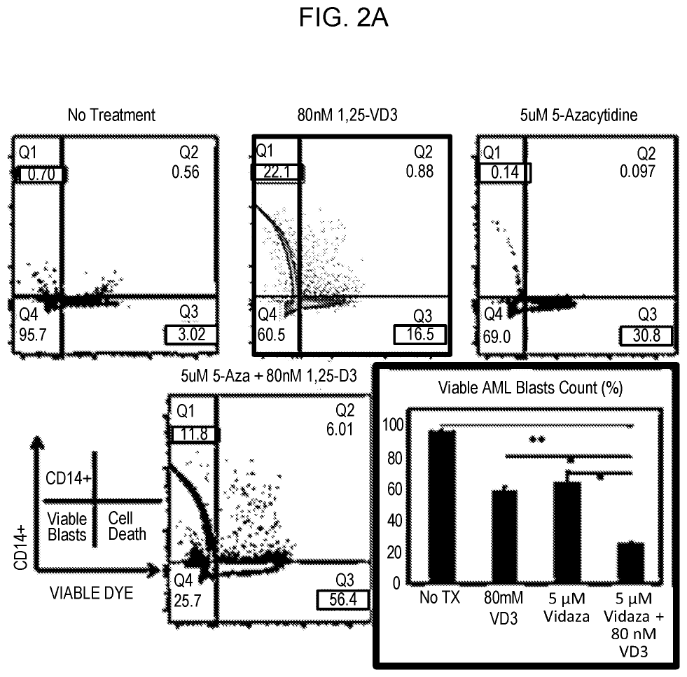

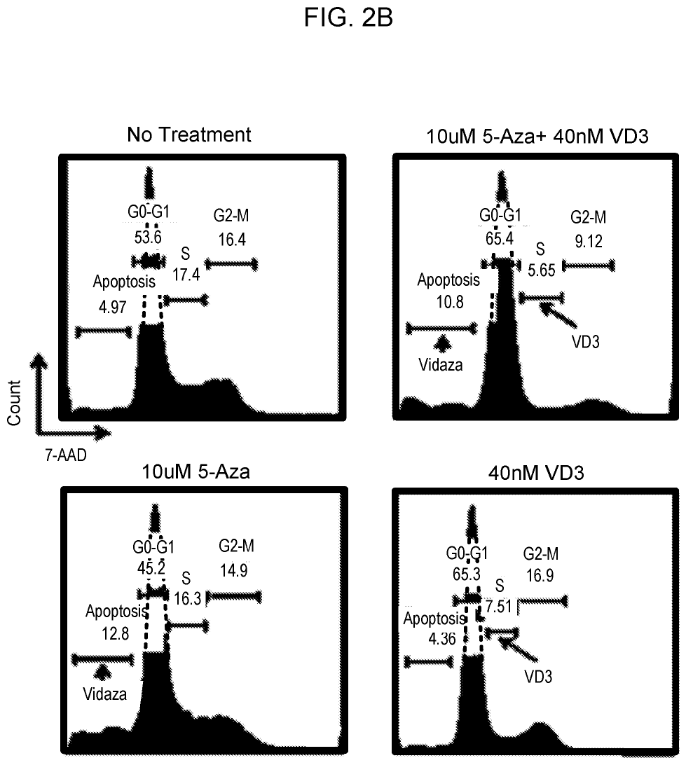

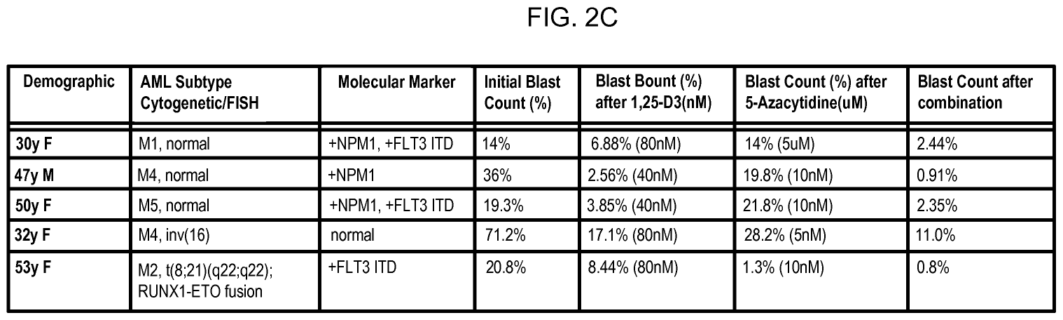

Additionally, it has been surprisingly found that the chemotherapeutic agent 5-azacytidine (AZA) and active VD3 work synergistically on leukemia cells in vitro and ex vivo. See, e.g., FIGS. 2A-2C. Thus, in one aspect the present disclosure provides compositions and methods in which AZA is used to condition bone marrow prior to the administration of the engineered hematopoietic stem cells.

### II. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

As used herein, “1α-hydroxylase” refers to 25-hydroxyvitamin D-1 alpha hydroxylase. 1α-hydroxylase is an enzyme that catalyzes the conversion of 25-hydroxyvitamin D3 (25(OH)D) to 1,25-dihydroxyvitamin D3 (1,25(OH)₂D). The gene that encodes 1α-hydroxylase is a human cytochrome P450 family 27 subfamily B member 1 (CYP27B1). Sequences for human CYP27B1 mRNA and 1α-hydroxylase protein are set forth in, e.g., NCBI GenBank Accession Nos. NM_000785.3 and NP_000776.1, respectively. In some embodiments, a hematopoietic stem cell as described herein is engineered to express a 1α-hydroxylase protein that has at least 70%, at least 75% at least 80%, at least 85%, at least 90%, at least 91%, at least 92%, at least 93%, at least 94%, at least 95%, at least 96%, at least 97%, at least 98%, or at least 99% identity to the 1α-hydroxylase protein set forth in NCBI GenBank Accession No. NP_000776.1.

The terms “identical” or “percent identity,” in the context of two or more polynucleotide or polypeptide sequences, refer to two or more sequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity) over a specified region. Methods for comparing polynucleotide or polypeptide sequences and determining percent identity are described in the art. See, e.g., Roberts et al., BMC Bioinformatics, 7:382, 2006, incorporated by reference herein.

The terms “nucleic acid” and “polynucleotide” are used interchangeably herein and refer to deoxyribonucleotides or ribonucleotides and polymers thereof in either single- or double-stranded form, and complements thereof. In some embodiments, the polynucleotide is DNA (e.g., genomic DNA or cDNA). In some embodiments, the polynucleotide is RNA (e.g., mRNA). Unless otherwise indicated, a particular nucleic acid sequence also implicitly encompasses conservatively modified variants thereof (e.g., degenerate codon substitutions), polymorphic variants (e.g., SNPs), splice variants, and nucleic acid sequences encoding truncated forms of proteins, complementary sequences, as well as the sequence explicitly indicated.

The terms “protein” and “polypeptide” are used interchangeably herein and refer to a polymer of amino acid residues. As used herein, the terms encompass amino acid chains of any length, including full-length proteins and truncated proteins.

The term “promoter,” as used herein, refers to a polynucleotide sequence capable of driving transcription of a coding sequence in a cell. In some embodiments, a promoter includes cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (e.g., turn on/off, regulate, modulate) gene transcription. A “constitutive promoter” is one that is capable of initiating transcription in nearly all tissue types, whereas a “tissue-specific promoter” initiates transcription only in one or a few particular tissue types.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., the promoter is from a different gene in the same species).

As used herein, a “subject” is a mammal, in some embodiments, a human. Mammals can also include, but are not limited to, farm animals (e.g., cows, pigs, horses, chickens, etc.), sport animals, pets, primates, horses, dogs, cats, mice and rats.

As used herein, the terms “treatment,” “treating,” and “treat” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; making the final point of degeneration less debilitating; and/or improving a subject's physical or mental well-being.

As used herein, a “therapeutic amount” or a “therapeutically effective amount” of an agent (e.g., an engineered hematopoietic stem cell, population of engineered hematopoietic stem cells, or pharmaceutical composition comprising an engineered hematopoietic stem cell as described herein) is an amount of the agent that prevents, alleviates, abates, or reduces the severity of symptoms of a disease (e.g., acute myeloid leukemia) in a subject. For example, for the given parameter, a therapeutically effective amount will show an increase or decrease of therapeutic effect of at least 5%, 10%, 15%, 20%, 25%, 40%, 50%, 60%, 75%, 80%, 90%, or at least 100%. Therapeutic efficacy can also be expressed as “fold” increase or decrease. For example, a therapeutically effective amount can have at least a 1.2-fold, 1.5-fold, 2-fold, 5-fold, or more effect over a control.

The terms “administer,” “administered,” or “administering,” as used herein, refer to introducing an agent (e.g., an engineered hematopoietic stem cell, population of engineered hematopoietic stem cells, or pharmaceutical composition comprising an engineered hematopoietic stem cell as described herein) into a subject or patient, such as a human. As used herein, the terms encompass both direct administration, (e.g., self-administration or administration to a patient by a medical professional) and indirect administration (e.g., the act of prescribing a compound or composition to a subject).

As used herein, the term “pharmaceutical composition” refers to a composition suitable for administration to a subject. In general, a pharmaceutical composition is sterile, and preferably free of contaminants that are capable of eliciting an undesirable response with the subject. Pharmaceutical compositions can be designed for administration to subjects in need thereof via a number of different routes of administration, including oral, intravenous, buccal, rectal, parenteral, intraperitoneal, intradermal, intratracheal, intramuscular, subcutaneous, inhalational, and the like.

### III. Engineered Hematopoietic Stem Cells

In one aspect, the present disclosure provides hematopoietic stem cells (HSCs) that are engineered to express or overexpress a polynucleotide that encodes a 1α-hydroxylase protein. In some embodiments, the HSC comprises a heterologous polynucleotide that encodes an 1α-hydroxylase protein. In some embodiments, the HSC comprises a heterologous expression cassette that comprises a polynucleotide that encodes an 1α-hydroxylase protein.

For the engineered HSCs of the present disclosure, the cell can be obtained or derived from any suitable source. In some embodiments, the HSC is a bone marrow-derived cell. In some embodiments, the HSC is a cord blood-derived cell. In some embodiments, the HSC is a peripheral blood-derived cell. Methods of isolating and generating HSCs are known in the art. See, e.g., Horwitz, 2007, “Sources of Human and Murine Hematopoietic Stem Cells,” Current Protocols in Immunology, 79:A:22A:2:22A.2.1-22A.2.6.

In some embodiments, the HSC is derived from a human subject. In some embodiments, the HSC is derived from a non-human mammal, e.g., a mouse. In some embodiments, the HSC is autologous to a subject (e.g., a subject to be administered the engineered HSC for the treatment of a leukemia). In some embodiments, the HSC is allogeneic to the subject. In some embodiments, the HSC is obtained from a subject that has been administered a chemotherapeutic agent, e.g., 5-azacytidine. For example, in some embodiments, HSCs are obtained from a subject (e.g., a human subject or a non-human mammal) following induction therapy with a chemotherapeutic agent, e.g., 5-azacytidine, and recovery of peripheral blood count. Methods for obtaining HSCs are known in the art. For example, HSCs can be obtained through bone marrow aspiration or through apheresis of mobilized peripheral blood cells.

In some embodiments, the polynucleotide encodes an 1α-hydroxylase protein that is human cytochrome P450 family 27 subfamily B member 1 (CYP27B1) (e.g., the polynucleotide sequence of NCBI GenBank Accession No. NM_000785.3 or a variant thereof). In some embodiments, the polynucleotide encodes a human 1α-hydroxylase protein having the sequence of the 1α-hydroxylase protein set forth in NCBI GenBank Accession No. NP_000776.1, or a variant thereof (e.g., a protein that has at least 70%, at least 75% at least 80%, at least 85%, at least 90%, at least 91%, at least 92%, at least 93%, at least 94%, at least 95%, at least 96%, at least 97%, at least 98%, or at least 99% identity to the 1α-hydroxylase protein set forth in NCBI GenBank Accession No. NP_000776.1).

In some embodiments, the polynucleotide that encodes a human CYP27B1 protein is operably linked to a promoter. In some embodiments, the promoter is a constitutively active promoter. Examples of suitable promoters include, but are not limited to, a spleen focus-forming virus (SFFV) promoter, a phosphoglycerate kinase (PGK) promoter, EF1a promoter, a cytomegalovirus (CMV) promoter, a Rous sarcoma virus promoter, a simian virus 40 (SV40) early promoter, a mouse mammary tumor virus promoter, a Moloney virus promoter, an avian leukemia virus promoter, or an Epstein-Barr virus immediate early promoter. In some embodiments, the promoter is a SFFV promoter, a PGK promoter, an EF1α promoter, or a CMV promoter. In some embodiments, the promoter is an inducible promoter (e.g., a tetracycline-inducible promoter). In some embodiments, the promoter is a tissue-specific promoter (e.g., a hematopoietic cell-specific promoter).

In some embodiments, the engineered HSC comprises an expression cassette that comprises a promoter operably linked to a heterologous polynucleotide that encodes the 1α-hydroxylase protein (e.g., a constitutively active promoter operably linked to a polynucleotide comprising a human CYP27B1 polynucleotide sequence such as the polynucleotide sequence of NCBI GenBank Accession No. NM_000785.3). In some embodiments, the engineered HSC comprises a vector that comprises an expression cassette that comprises a promoter operably linked to a heterologous polynucleotide that encodes the 1α-hydroxylase protein.

In some embodiments, the polynucleotide that encodes the 1α-hydroxylase protein is expressed in the HSC using a virus or viral vector. In some embodiments, the virus is an adenovirus, lentivirus, adeno-associated virus, or retrovirus. In some embodiments, the virus is a lentivirus. Viruses and viral vectors containing the polynucleotide that encodes the 1α-hydroxylase protein can be introduced into the HSC by methods known in the art, such as but not limited to, transfection, electroporation, microinjection, transduction, cell fusion, DEAE dextran, calcium phosphate precipitation, or lipofection.

In some embodiments, the engineered HSC (e.g., an engineered HSC comprising an expression cassette or vector as disclosed herein) overexpresses the 1α-hydroxylase protein, as compared to a HSC lacking the heterologous polynucleotide. In some embodiments, the engineered HSC comprising a heterologous polynucleotide expresses the 1α-hydroxylase protein at a level that is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, or at least 50-fold higher than an HSC lacking the heterologous polynucleotide.

Protein expression can be detected and quantified using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). In some embodiments, protein expression is detected by quantitative mass spectrometry, for example but not limited to, spectral count MS, ion intensities MS, metabolic labeling (e.g., stable-isotope labeling with amino acids in cell culture (SILAC), enzymatic labeling, isotopic labeling (e.g., isotope-coded protein labeling (ICPL) or isotope-coded affinity tags (ICAT)), and isobaric labeling (e.g., tandem mass tag (TMT)).

In some embodiments, an engineered HSC that overexpresses the 1α-hydroxylase protein (e.g., an engineered HSC comprising an expression cassette or vector as disclosed herein) produces a higher amount or concentration of active 1,25-dihydroxyvitamin D3 (VD3) than an HSC lacking the heterologous polynucleotide. For example, in some embodiments, an engineered HSC that overexpresses the 1α-hydroxylase protein produces an amount or concentration of active VD3 that is at least 10-fold, at least 15-fold, at least 20-fold, at least 25-fold, at least 30-fold, at least 40-fold, at least 50-fold, at least 60-fold, at least 70-fold, at least 80-fold, at least 90-fold, or at least 100-fold more than the amount or concentration of active VD3 that is produced by an HSC lacking the heterologous polynucleotide. In some embodiments, production of active VD3 is measured by culturing a cell (e.g., an engineered HSC that overexpresses the 1α-hydroxylase protein) in the presence of inactive VD3 (25(OH)-D3) for a period of time, e.g., 24 hours, 48 hours, or 72 hours, and subsequently quantitatively analyzing the cell supernatant for 1,25(OH)2-D3. Methods of quantitatively analyzing the cell supernatant for 1,25(OH)2-D3 are known in the art. In some embodiments, mass spectrometry or liquid chromatography-mass spectrometry is used for quantitatively analyzing the cell supernatant for 1,25(OH)2-D3. In one embodiment, active VD3 production is measured according to the method disclosed in the Examples section below.

In some embodiments, the engineered HSC is expanded ex vivo in order to form a population of engineered HSCs. Methods for expanding HSCs are described in the art. See, e.g., Kumar et al., Trends Mol Med, 2017, 23:799-819. In some embodiments, the engineered HSCs are expanded in the presence of an expansion medium, e.g., Stemline II Hematopoietic Stem Cell Expansion Medium (Sigma). In some embodiments, the expansion occurs in the presence of one or more growth factors or cytokines (e.g., in an expansion medium supplemented with one or more growth factors or cytokines).

In some embodiments, the engineered HSC or population of engineered HSCs is stimulated with one or more cytokines or chemotactic factors. Without being bound to a particular theory, it is believed that treating HSCs with a cytokine or chemotactic factor released in the bone marrow can improve the efficiency of HSC homing to bone marrow and subsequent engraftment. Suitable chemotactic factors include, but are not limited to, α-chemokine stromal-derived factor 1 (SDF-1), the bioactive phosphosphingolipids sphingosine-1-phosphate (S1P) and ceramid-1-phosphate (C1P). Suitable cytokines include, but are not limited to, stem cell factor (SCF), IL-3, IL-6, and IL-11. In some embodiments, the engineered HSC or population of engineered HSCs is treated with SCF and/or IL-3.

In some embodiments, compositions comprising an engineered HSC or population of engineered HSCs as described herein are provided. In some embodiments, the composition further comprises a pharmaceutically acceptable excipient. Guidance for preparing formulations for use in the present invention is found in, for example, Remington: The Science and Practice of Pharmacy, 21st Edition, Philadelphia, Pa. Lippincott Williams & Wilkins, 2005.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, supra. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

For administration by injection or infusion, the engineered HSC or population of engineered HSCs can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, stabilizers and preservatives. In some embodiments, an aqueous solution is used, such as a physiologically compatible buffer such as Hanks's solution, Ringer's solution, or physiological saline buffer. Formulations can be presented in unit dosage form, e.g., in ampules or in multi-dose containers, with an added preservative.

### IV. Methods of Treatment

In another aspect, therapeutic methods for the treatment of leukemia, such as AML, are provided. In some embodiments, a method of treating a human subject having leukemia is provided. In some embodiments, the method comprises administering to the subject a population of engineered hematopoietic stem cells or a pharmaceutical composition comprising a population of engineered hematopoietic stem cells, wherein the engineered hematopoietic stem cells comprise a heterologous expression cassette that comprises a promoter operably linked to a polynucleotide that encodes a human CYP27B1 protein. In some embodiments, the therapeutic methods comprise administering an engineered HSC, population of engineered HSCs, or pharmaceutical composition as disclosed in Section III above.

In some embodiments, the subject to be treated has AML. It will be recognized by a person of ordinary skill in the art that AML is classified into subtypes according to one of two classification systems, the French-American-British (FAB) classification and the World Health Organization (WHO) classification. In some embodiments, the subject has one of the following subtypes of AML, as classified by the FAB classification: undifferentiated acute myeloblastic leukemia (M0), acute myeloblastic leukemia with minimal maturation (M1), acute myeloblastic leukemia with maturation (M2), acute promyelocytic leukemia (APL) (M3), acute myelomonocytic leukemia (M4), acute myelomonocytic leukemia with eosinophilia (M4 eos), acute monocytic leukemia (M5), acute erytroid leukemia (M6), or acute megakaryocytic leukemia (M7), as classified by the FAB classification. In some embodiments, the subject has one of AML subtypes M0, M1, M2, M4, M5, M6, or M7.

In some embodiments, the subject has one of the following subtypes of AML, as classified by the WHO classification: AML with a genetic abnormality (e.g., a translocation between chromosomes 8 and 21, a translocation or inversion in chromosome 16, a translocation between chromosomes 9 and 11, a translocation between chromosomes 15 and 17, a translocation between chromosomes 6 and 9, a translocation or inversion in chromosome 3, or a translocation between chromosomes 1 and 22), AML with myelodysplasia-related changes, AML related to previous chemotherapy or radiation, AML not otherwise specified (e.g., AML with minimal differentiation, AML without maturation, AML with maturation, acute myelomonocytic leukemia, acute monocytic leukemia, acute erythroid leukemia, acute megakaryoblastic leukemia, acute basophilic leukemia, or acute panmyelosis with fibrosis), myeloid sarcoma, myeloid proliferations related to Down syndrome, or undifferentiated and biphenotypic acute leukemias (also referred to as AML with lymphoid markers or mixed phenotype acute leukemias).

In some embodiments, the subject has a form of AML that comprises one or more mutations in the gene encoding the receptor tyrosine kinase FLT3. Mutations in FLT3, such as internal tandem duplications (ITD) or point mutations, are found in about a third of all AML patients. Small, Hematology Am Soc Hematol Educ Program, 2006, 1:178-184. In some embodiments, the AML comprises an internal tandem duplication in the FLT3 gene (ITD-FLT3) and/or a point mutation in the FLT3 gene.

In some embodiments, the subject to be treated is a human. In some embodiments, the subject is an adult. In some embodiments, the subject is a juvenile.

In some embodiments, the subject to be treated has previously been treated with 5-azacytidine (AZA). In some embodiments, the subject to be treated has previously been treated with AZA within 1, 2, 3, 4, 5, 6, or 7 days of administering the engineered hematopoietic stem cells. In some embodiments, the subject is treated with AZA at a dosage that is suitable for induction therapy. In some embodiments, the subject is treated with AZA at a dosage that is suitable for maintenance therapy. Suitable dosages for AZA therapy can be readily determined by a person of ordinary skill in the art.

In some embodiments, the engineered hematopoietic stem cells that are administered to the subject have been stimulated with one or more cytokines or chemotactic factors. For example, in some embodiments, the engineered hematopoietic stem cells are stimulated with a chemotactic factor such as, but not limited to, α-chemokine stromal-derived factor 1 (SDF-1), sphingosine-1-phosphate (S1P), or ceramid-1-phosphate (C1P). In some embodiments, the engineered hematopoietic stem cells are stimulated with a cytokine such as, but not limited to, stem cell factor (SCF), IL-3, IL-6, and IL-11. In some embodiments, the engineered hematopoietic stem cells are stimulated with SCF and/or IL-3.

In some embodiments, the subject is administered engineered hematopoietic stem cells that are autologous to the subject. In some embodiments, the subject is administered engineered hematopoietic stem cells that are allogeneic to the subject. In some embodiments, hematopoietic stem cells are obtained from the subject to be treated after the subject has been administered a chemotherapeutic agent, e.g., 5-azacytidine. For example, in some embodiments, hematopoietic stem cells are obtained from the subject following induction therapy with a chemotherapeutic agent, e.g., 5-azacytidine, and subsequent recovery of peripheral blood count. In some embodiments, hematopoietic stem cells are obtained from the subject (e.g., following induction therapy with a chemotherapeutic agent), the cells are engineered as disclosed herein to express a polynucleotide that encodes a 1α-hydroxylase protein, then the engineered cells are expanded ex vivo in order to form a population of engineered hematopoietic stem cells that are subsequently administered to the subject.

The route of administration of the engineered HSCs or pharmaceutical composition comprising engineered HSCs (e.g., as described in Section III above) can be oral, intraperitoneal, transdermal, subcutaneous, intravenous, intramuscular, inhalational, topical, intralesional, rectal, intrabronchial, intralymphatic, intradermal, nasal, transmucosal, intestinal, ocular or otic delivery, or any other methods known in the art. In some embodiments, an engineered HSC, population of engineered HSCs, or pharmaceutical composition comprising an engineered HSC as described herein is administered by intravenous injection or by subcutaneous injection. In some embodiments, an engineered HSC, population of engineered HSCs, or pharmaceutical composition comprising an engineered HSC as described herein is administered systemically. In some embodiments, an engineered HSC, population of engineered HSCs, or pharmaceutical composition comprising an engineered HSC as described herein is administered locally.

Dosages and desired concentrations of the engineered HSCs of the disclosure may vary depending on the particular use envisioned. The determination of the appropriate dosage or route of administration is well within the skill of one in the art. Typically the amount of the cells and/or compositions administered to a subject is a therapeutically effective amount. In some embodiments, a therapeutically effective amount of engineered HSCs or composition comprising engineered HSCs is an amount that prevents or reverses one or more symptoms of AML. For example, in some embodiments, a therapeutically effective amount is at least about 100, 500, 1,000, 2,500, 5,000, 10,000, 20,000, 50,000, 100,000, 500,000, 1,000,000, 5,000,000, or 10,000,000 cells or more (e.g., per administration). In some embodiments, a therapeutically effective amount of the engineered HSCs or composition comprising engineered HSCs is administered about once per day, once per week, twice per week, once per month, or twice per month.

The engineered HSCs, populations of engineered HSCs, and compositions comprising engineered HSCs may be administered to a subject in need thereof for a predetermined time, an indefinite time, or until an endpoint is reached. In some embodiments, treatment is continued on a continuous daily or weekly basis for at least two to three months, six months, one year, or longer. In some embodiments, treatment is for at least 30 days, at least 60 days, at least 90 days, at least 120 days, at least 150 days, or at least 180 days. In some embodiments, treatment is continued for at least 6 months, at least 7 months, at least 8 months, at least 9 months, at least 10 months, at least 11 months, or at least one year. In some embodiments, treatment is continued for the rest of the patient's life or until administration is no longer effective to provide meaningful therapeutic benefit.

### V. Kits

In another aspect, kits comprising the engineered HSC, population of engineered HSCs, or pharmaceutical composition comprising an engineered HSC or population of engineered HSCs as described herein are provided. In some embodiments, the kit comprises an engineered HSC, population of engineered HSCs, or pharmaceutical composition as disclosed in Section III above.

In some embodiments, the kit further comprises instructional materials containing directions (i.e., protocols) for the practice of the methods of this disclosure (e.g., instructions for using the kit for treating AML). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

### VI. Examples

The following examples are offered to illustrate, but not to limit, the claimed invention.

### Example 1: Differentiation of AML Cells in Response to Cell-Mediated Delivery of CYP27B1

This example describes the transduction of an AML cell line with a lentiviral vector expressing the gene CYP27B1, which encodes 25-OH-D3 1-α-hydroxylase, an enzyme that converts 25[OH]D (calcidiol) into 1,25[OH]₂D (calcitriol).

Preparing the plasmids: The 1.6 kb mouse CYP27B1 (mCYP27B1) cDNA fragment with a 5′ KOZAK ribosome entry sequence was cloned into the modified pRSC-SFFV-Luciferase-E2A-GFP-WPRE lentiviral vector. The resulting construct was designated SFFV-CYP27B1-Luciferase-E2A-GFP (“lenti-CYP-Luc-GFP”).

Lentivirus transduction: 1×10⁶ cells/well were cultured in a total volume of 0.5 ml culture medium containing 50 μl virus (MOI=20) and 8 μg/ml protamine in a 6-well plate. Twenty-four hours later, the virus was removed and culture media replenished. The cells were cultured for another 24 hours and examined for transduction efficiency under a fluorescence microscope. If necessary, the above transduction procedure was repeated one more time.

Test for functionality of the CYP-GFP-C2C12 and CYP-GFP-MOLM-14: A range of 25(OH)-D3 (inactive VD3) concentrations was added to the cultured cells for 3 days (72 hours) as shown in Table 1 below. Cell supernatants were collected and quantitatively analyzed for 1,25(OH)₂-D3 (active VD3) using liquid chromatography-mass spectrometry (Heartland Assays). The results of the active VD3 production assay are shown in Table 1 below.

Terminal differentiation capability of AZA/1,25-D3 combination therapy: Terminal differentiation is used to measure the expression of CD14, which a surface marker for mature monocytes. Flow cytometry was used to quantify CD14 marker expression.

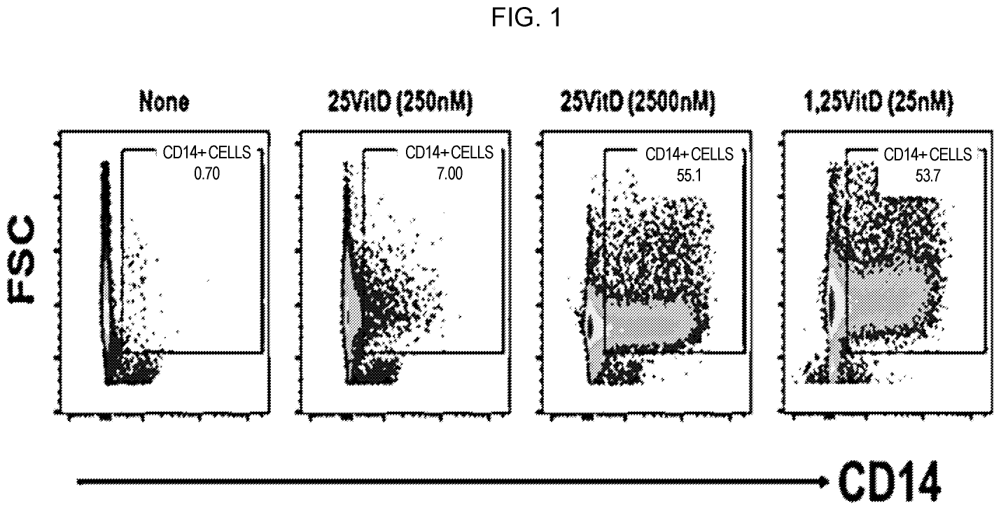

Results: The MOLM14 (human AML leukemia) and C2C12 (mouse mesenchymal progenitor) cell lines were successfully transduced with CYP27B1-GFP lenti-viral vector with high efficiency (98.8% and 95.6%, respectively). These cells were able to produce high concentrations of active VD3 (see Table 1 below; normal human serum level is 0.1 nM) in co-cultures supplemented with inactive (25-OH-D3). Further, as shown in FIG. 1, the supernatant collected from the co-culture induced terminal differentiation of MOLM14 AML cells.

TABLE 1
Active VD3 production in CYP-GFP-
C2C12 and CYP-GFP-MOLM-14 cells

| 25(OH)-D3 | 1,25(OH)2-D3 | 25(OH)-D3 | 1,25(OH)2-D3 |
|---|---|---|---|
| (nM) | (nM) | (nM) | (nM) |
| 0 | 0.188 | 0 | <1.2 |
| 25 | 2.43 | 500 | 55.49 |
| 250 | 41.35 | 1000 | >76.92 |
| 2500 | >76.92 | 2000 | >76.92 |

### Example 2: Synergistic Anti-Leukemia Effects of AZA in Combination with Active VD3

This example describes data demonstrating that AZA and active VD3 work synergistically on leukemic cells both in vitro and ex vivo.

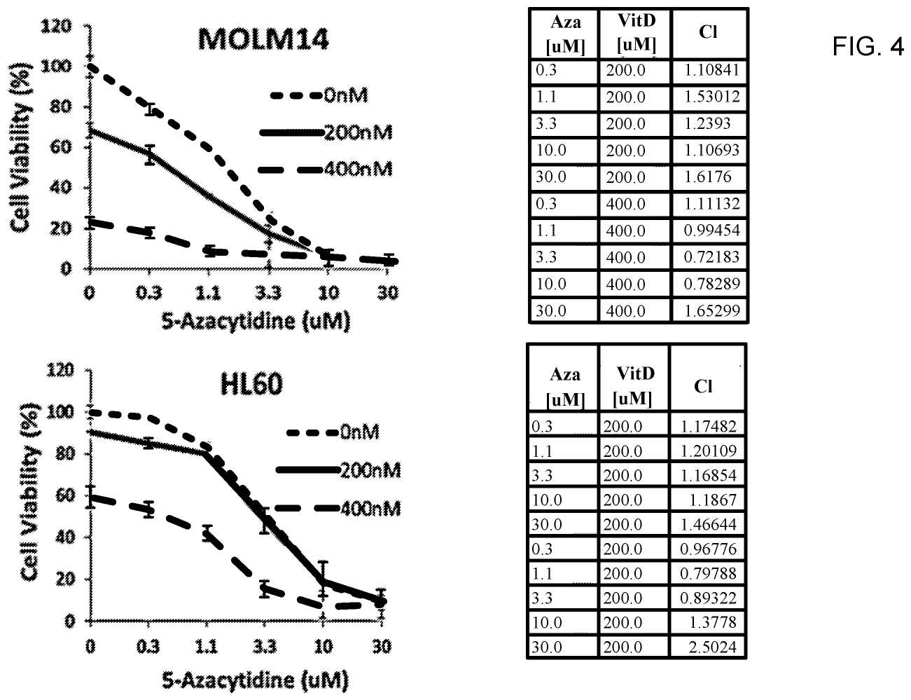

An MTT reduction assay of treated MOLM14 cells with a combination of VD3 and AZA was performed to evaluate synergy between VD3 and AZA. HL-60 and MOLM-14 cells were cultured in 96-well plates and treated with various combinations of VIDAZA® (azacytidine) and active VD3 for 48 hours. The MTT assay was used to measure the antiproliferative effects of treatment and the Combination Index (CI) was calculated to evaluate synergy (synergy=CI<1). As shown in FIG. 4, the MTT reduction assay resulted in a CI of less than 1 for various concentrations, which indicates a synergy between VD3 and AZA. This synergistic effect was further confirmed by flow cytometry in which treated MOLM14 cells were stained with viability dye and CD14 (marker of AML cells that have differentiated to monocytes) (FIG. 2A).

Studies to elucidate the mechanisms of combination treatment were conducted. MOLM14 cells were treated with different concentration of VIDAZA®, VD3, and their combination for 48 hours. After 48 hours of treatment, cells were stained with 7-AAD, a fluorescent compound that stains DNA, which is typically used for cell cycle studies. As shown in FIG. 2B, VIDAZA® caused significant apoptosis, while VD3 inhibited DNA synthesis. In addition, the combination treatment of VIDAZA® and VD3 resulted in increase in both apoptosis and inhibition of DNA synthesis, as shown in FIG. 2B.

Ex vivo experiments were performed to examine the effect of 1,25-D3/AZA combination therapy. Five patients' leukapheresis samples were obtained from the Loma Linda Biospecimen Laboratory. The patients' data including flow cytometry, cytogenetic, fluorescent in situ hybridization (FISH) and molecular markers were reviewed and organized in the top table. Each patient sample was then treated with different concentrations of 1,25-D3 alone, AZA alone and 1,25-D3/AZA combination. After 48 hours of treatment, collected cells were stained with viable dye, serials of blast CD markers and were analyzed by flow cytometry. As shown in FIG. 2C, in the five primary AML patient samples tested, superior efficacy of combination treatment, as compared to single treatment, was observed.

### Example 3: HSC-Mediated Delivery of Therapeutic VD3 to Bone Marrow

This example describes how hematopoietic stem cells (HSCs) can be used as a vehicle to deliver therapeutic doses of VD3 to the bone marrow (BM) for the treatment of AML.

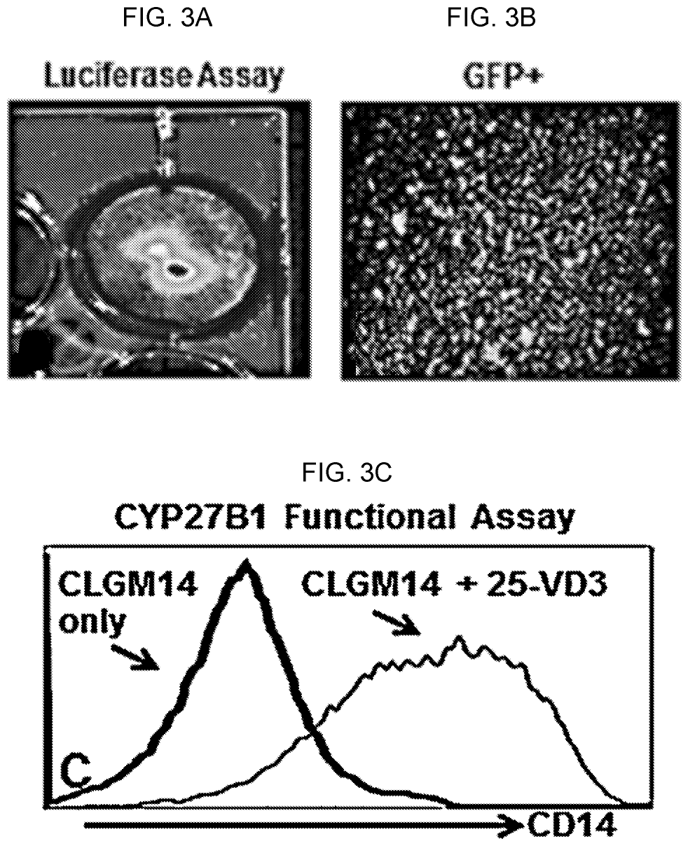

A CYP27B1-luciferase-GFP lentiviral vector was constructed. The 1.6 kb mouse CYP27B1 (mCYP27B1) cDNA fragment with a 5′ KOZAK ribosome entry sequence was cloned into the modified pRSC-SFFV-Luciferase-E2A-GFP-WPRE lentiviral vector. The resulting construct was designated SFFV-CYP27B1-Luciferase-E2A-GFP (“lenti-CYP-Luc-GFP”). This vector was tested as shown in FIGS. 3A-3C.

HSCs are transduced with the CYP27B1-luciferase-GFP lentiviral vector. To evaluate the homing and expansion of engineered HSCs, in vivo bioluminescence imaging is used to quantitate HSCs localized in the BM for up to three weeks. Expansion of HSC progeny expressing CYP27B1 (CD45+ GFP+ RFP−) is confirmed by flow cytometry analysis of BM harvested from groups of animals euthanized at weekly time points for up to three weeks.

For therapeutic treatment, the subject (e.g., a human or a non-human mammal) is pre-treated with AZA chemotherapy to condition BM for homing and retention of engineered HSCs. Engineered HSCs are injected 24 hours after chemotherapy.

To optimize the numbers of CYP27B1-HSCs that are infused for therapeutic treatment, serum calcium level can be used as a marker. 1E5 HSCs are initially infused into the subject and serum calcium level is measured within 96 hours. Serum calcium levels can be measured using the Calcium Colorimetric Assay Kit (BioVision, Milpitas, Calif.). If no hypercalcemia is detected, the subjects (e.g., mice) are infused with increasing numbers of HSCs, in increments of 100,000, up to 1 million cells per animal until hypercalcemia is detected. The optimal number is the highest number, up to one million, that does not cause hypercalcemia.

To determine if the local concentration of VD3 is sufficient to differentiate leukemic blasts, the amount of differentiated blasts can be measured at a defined period of time after treatment. For example, for mice that are administered CYP27B1-HSCs, following one week of treatment the mice can be euthanized and their BM harvested for assessment of differentiated blasts (CD14+ RFP+) by flow cytometry.

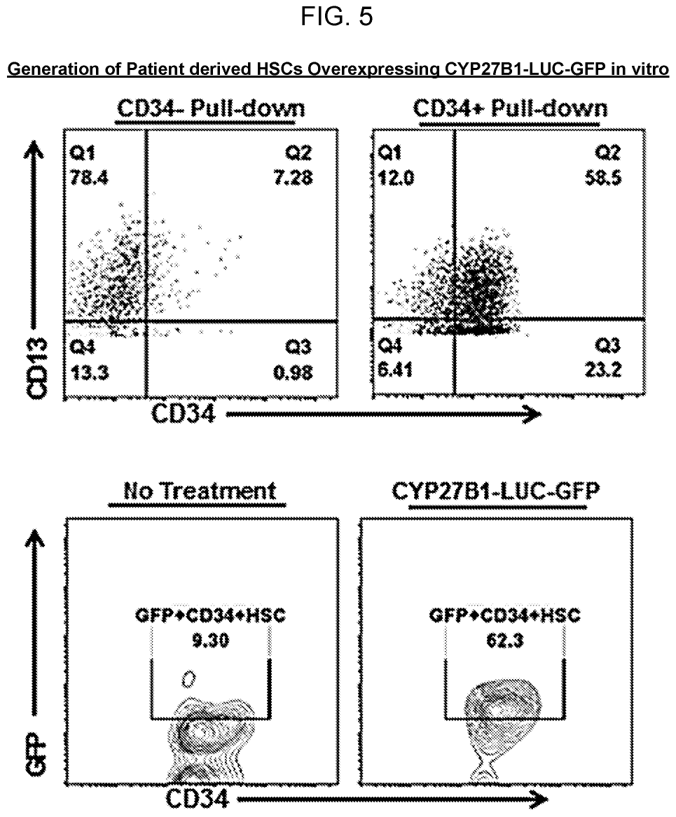

In one experiment, human CD34+ cells were isolated from AML patient peripheral blood using CD24 MicroBead Kit and MACS Separator (Miltenyi Biotec), according to the manufacturer's protocol. These CD34+ cells were cultured for one day and then received FACS analysis. As shown in FIG. 5, after confirming the high population of CD34+ HSCs in vitro, CYP27B1-LUC-GFP viral transduction was performed. Using FACS analysis it was found that 62.3% of the cells were CYP-GFP+CD34+ HSCs. These cells were then expanded in Stemline II Hematopoietic Stem Cell Expansion Medium (Sigma).

### Example 4: 1,25 Active Vitamin D Works on Both FLT3-ITD AML Cell Lines and Ex Vivo FLT3-ITD Patient Primary Cells

FLT3 is one of the key molecules with a role in the pathogenesis in AML. (ITD) Internal tandem duplications or point mutation of the receptor tyrosine kinase (RTK) FLT3 is found in one third of cases with acute myeloid leukemia (AML). This genetic aberration may lead to the constitutive activation of the receptor, thus providing the molecular basis for a persisting growth stimulus with worse clinical outcomes.

To elucidate the role of Vitamin D in precise therapies for subsets of AML, we screened 4 AML-derived cell lines in in vitro studies. Two cell lines contained ITD of the FLT3 gene, including MV4-11 cell line with exclusively the mutated allele and MOLM-14 cell line with a mutated and the wild-type version of the gene. In addition, two AML cell lines without FLT3 mutations such as HL-60 and THP-1 were included in the study.

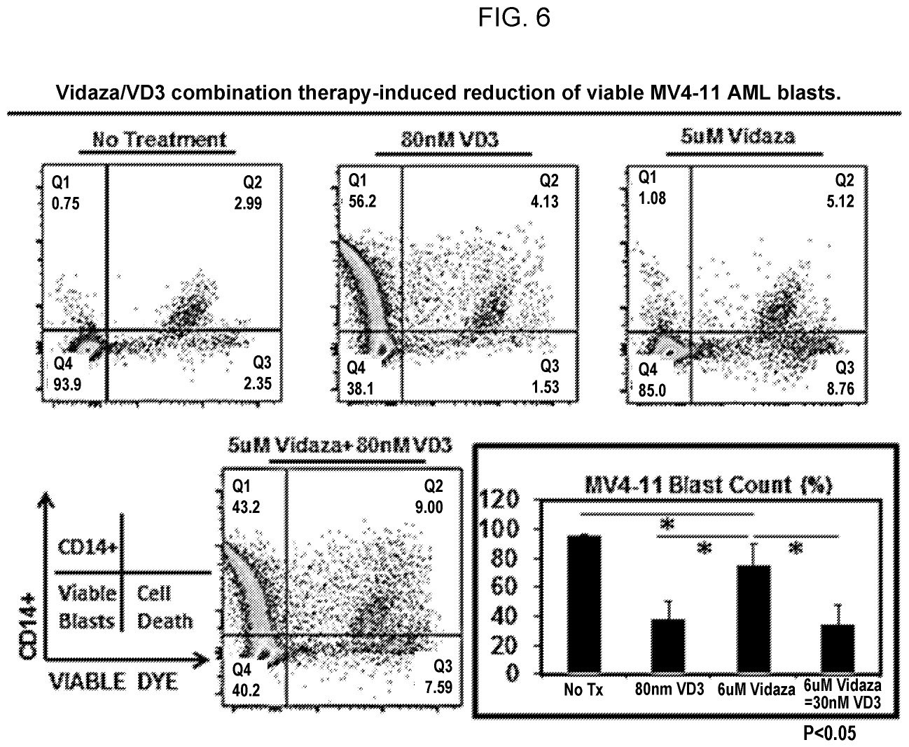

MV4-11 AML cell line was treated with different concentration of VIDAZA®, VD3, and their combination for 48 hours. After 48 hours of treatment, cells were stained with viable dye and CD14 markers. CD14 is a marker for maturation of monoblasts. As shown in FIG. 6, comparison data demonstrated that 80 nM 1,25-D3+AZA combination therapy significantly reduced the percentage of viable blasts (Viable/CD14− cells in the gating strategy) from 95.5% with no treatment (p<0.05), and 74.8% with 5 μM AZA alone (p<0.05) to 34.1%. Additionally, therapy with 80 nM 1,25-D3 alone reduced more blasts compared to 5 μM AZA alone by 38.5% vs 74.8% (p<0.05). No therapeutic effect of VIDAZA®/VD3 combination therapy was observed for THP-1 AML blasts (data not shown).

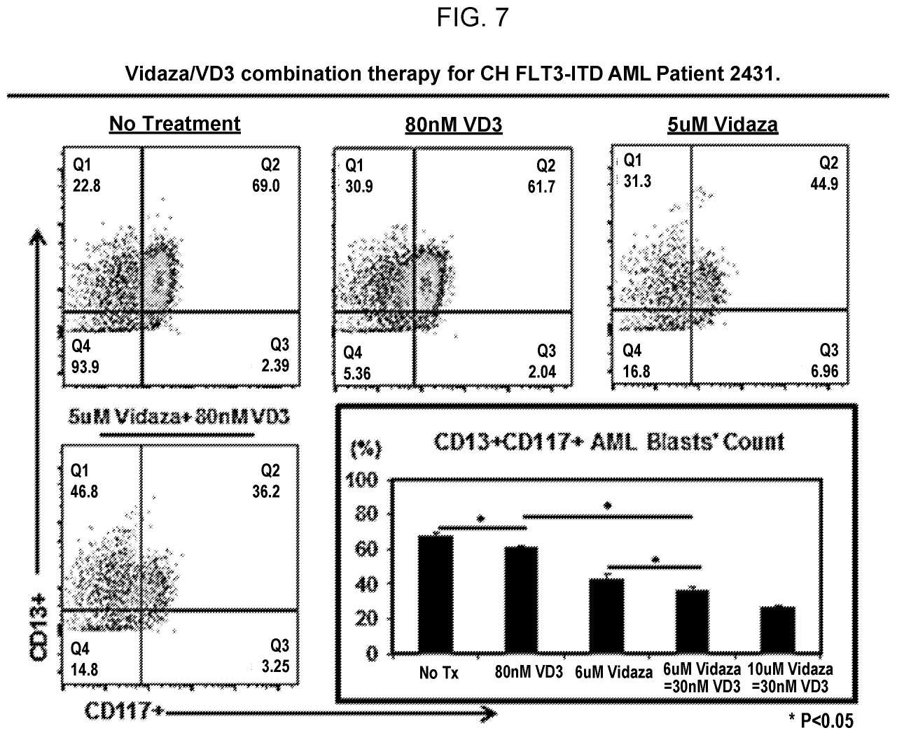

An ex vivo assay was performed to test VIDAZA®/VD3 combination therapy-induced CD13+CD117+ blast reduction in FLT3-ITD patient primary dells. A patient sample (patient 2431) was treated with different concentration of VIDAZA®, VD3, and their combination for 48 hrs. As shown in FIG. 7, after 48 hours of treatment, cells were stained with Viable dye and different blast CD markers. The combination of 5 μM VIDAZA®+80 nM VD3 showed the most significant reduction of blast cells compared to the controls, from 68.2% with no treatment (p<0.05), 61.8% with 80 nM 1,25-D3 alone (p<0.05), and 42.6% with 5 μM AZA alone to 36.6% (p<0.05).

All publications and patents cited in this specification are herein incorporated by reference as if each individual publication or patent were specifically and individually indicated to be incorporated by reference and are incorporated herein by reference to disclose and describe the methods and materials in connection with which the publications are cited.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

It should be understood that although the present invention has been specifically disclosed by certain aspects, embodiments, and optional features, modification, improvement, and variation of such aspects, embodiments, and optional features can be resorted to by those skilled in the art, and that such modifications, improvements, and variations are considered to be within the scope of this disclosure.

What is claimed is:

1. An engineered hematopoietic stem cell comprising a heterologous expression cassette, the expression cassette comprising a promoter operably linked to a polynucleotide that encodes a 1α-hydroxylase protein, wherein the 1α-hydroxylase protein is human cytochrome P450 family 27 subfamily B member 1 (CYP27B1).

2. The engineered hematopoietic stem cell of claim 1, wherein the promoter is a constitutively active promoter.

3. The engineered hematopoietic stem cell of claim 2, wherein the promoter is a SFFV promoter, a PGK promoter, an EF1α promoter, or a CMV promoter.

4. The engineered hematopoietic stem cell of claim 1, wherein the promoter is a tissue-specific promoter.

5. The engineered hematopoietic stem cell of claim 1, wherein the promoter is an inducible promoter.

6. (canceled)

7. (canceled)

8. (canceled)

9. (canceled)

10. The engineered hematopoietic stem cell of claim 1, wherein the engineered hematopoietic stem cell overexpresses CYP27B1.

11. The engineered hematopoietic stem cell of claim 1, wherein the engineered hematopoietic stem cell is stimulated with one or more cytokines.

12. (canceled)

13. (canceled)

14. (canceled)

15. A method of treating a human subject having a leukemia, the method comprising:
administering to the subject a population of engineered hematopoietic stem cells or a pharmaceutical composition comprising a population of engineered hematopoietic stem cells, wherein the engineered hematopoietic stem cells comprise a heterologous expression cassette that comprises a promoter operably linked to a polynucleotide that encodes a human CYP27B1 protein.

16. The method of claim 15, wherein the leukemia is acute myeloid leukemia (AML).

17. (canceled)

18. The method of claim 16, wherein the AML comprises a mutation in FLT3.

19. The method of claim 18, wherein the mutation in FLT3 comprises an internal tandem duplication in FLT3.

20. (canceled)

21. (canceled)

22. (canceled)

23. (canceled)

24. (canceled)

25. The method of claim 15, wherein the subject has previously been treated with 5-azacytidine within one day of administering the engineered hematopoietic stem cells.

26. The method of claim 15, wherein the engineered hematopoietic stem cells are autologous to the subject.

27. The method of claim 26, wherein the autologous cells are obtained from the subject after treatment with 5-azacytidine.

28. (canceled)

29. The method of claim 15, wherein the engineered hematopoietic stem cells are stimulated with one or more cytokines.

30. (canceled)

31. (canceled)
